(19) United States
(12) Patent Application Publication
(10) Pub. No.: US 2015/0305707 A1
(43) Pub. Date: Oct. 29, 2015

(54) ULTRASONIC DIAGNOSTIC APPARATUS, IMAGE PROCESSING APPARATUS, AND IMAGE PROCESSING METHOD

(71) Applicants: Kabushiki Kaisha Toshiba, Minato-ku (JP); Toshiba Medical Systems Corporation, Otawara-shi (JP)

(72) Inventors: Tomoya OKAZAKI, Kawasaki (JP); Yasuhiko ABE, Otawara (JP); Tomoyuki TAKEGUCHI, Kawasaki (JP); Yukinobu SAKATA, Kawasaki (JP)

(73) Assignees: Kabushiki Kaisha Toshiba, Minato-ku (JP); Toshiba Medical Systems Corporation, Otawara-shi (JP)

(21) Appl. No.: 14/695,565

(22) Filed: Apr. 24, 2015

(30) Foreign Application Priority Data
Apr. 25, 2014 (JP) 2014-091878
Mar. 25, 2015 (JP) 2015-063071

(51) Int. Cl. A61B 8/08; A61B 8/13; A61B 8/00

(52) U.S. Cl. CPC A61B 8/0883; A61B 8/463; A61B 8/13; A61B 8/466; A61B 8/5207

(57) ABSTRACT

According to an embodiment, an ultrasonic diagnostic apparatus includes an acquirer, first and second generators, and a display controller. The acquirer acquires a 3D ultrasonic image including a heart. The first generator generates a first cross-sectional image of the 3D ultrasonic image including a first axis passing through a second portion in the 3D ultrasonic image and including a third portion. The second portion is a portion by which one of blood inflow into a first portion which is an atrium or a ventricle and blood outflow from the first portion is performed. The third portion is a portion by which another of the blood inflow and the blood outflow is performed. The second generator generates a second cross-sectional image of the 3D ultrasonic image which includes a second axis passing through the third portion and which intersects with the first cross-sectional image. The display controller displays the cross-sectional images.

### CROSS-REFERENCE TO RELATED APPLICATIONS

This application is based upon and claims the benefit of priority from Japanese Patent Application No. 2014-091878, filed on Apr. 25, 2014 and Japanese Patent Application No. 2015-063071, filed on Mar. 25, 2015; the entire contents of which are incorporated herein by reference.

### FIELD

Embodiments described herein relate generally to an ultrasonic diagnostic apparatus, an image processing apparatus, and an image processing method.

### BACKGROUND

Conventionally, as a method of inputting a 3D boundary of an object included in a 3D medical image, there is known a technique of tracing the boundary of an object on a plurality of cross-sectional images and generating the 3D boundary by interpolating the cross-sections.

For example, in the case of inputting a 3D boundary of the myocardium (muscle that makes up the heart) of the left ventricle of the heart included in an ultrasonic medical image, there is known a technique of tracing the myocardial boundary on a plurality of short-axis cross-sections of the left ventricle and generating the 3D myocardial boundary by interpolating the cross-sections.

For example, if the target of analysis is only the main portion (atrium or ventricle) and the inflow portion (for example, the mitral valve in the case of the left ventricle, and the tricuspid valve in the case of the right ventricle) for the blood to flow into the main portion, the position of an ultrasonic probe is set in accordance with the axis passing through the main portion and the inflow portion, and both are displayed relatively clearly with only the short-axis cross-section according to the conventional technique.

However, in the case where the target of analysis includes, in addition to the main portion and the inflow portion, an outflow portion (for example, the pulmonary valve in the case of the right ventricle) for the blood to flow out of the main portion, if the position of the ultrasonic probe is set in accordance with the axis passing through the main portion and one of the inflow portion and the outflow portion, the visibility of one of the inflow portion and the outflow portion may be sufficiently secured, but the visibility of the other may not be sufficiently secured. As a result, the time taken for the analysis and the diagnosis is increased. Besides, it is difficult to appropriately set the myocardial boundary, and thus, the accuracy of the analysis and the diagnosis may not be sufficiently secured.

### BRIEF DESCRIPTION OF THE DRAWINGS

FIG. 1 is a diagram illustrating an example configuration of an ultrasonic diagnostic apparatus according to a first embodiment;

FIG. 2 is a diagram illustrating an example functional configuration of an image processor of the first embodiment;

FIG. 3 is a schematic diagram representing the axis of volume data of the first embodiment;

FIG. 4 is a schematic diagram representing a cross-sectional image of the first embodiment;

FIG. 5 is a schematic diagram of a heart included in the volume data of the first embodiment;

FIG. 6 is a schematic diagram representing a first cross-sectional image of the first embodiment;

FIG. 7 is a schematic diagram representing a second cross-sectional image of the first embodiment;

FIG. 8 is a diagram illustrating example processing performed by the image processor of the first embodiment;

FIG. 9 is a diagram illustrating an example functional configuration of an image processor of a modification of the first embodiment;

FIG. 10 is a diagram illustrating an example functional configuration of an image processor of a modification of the first embodiment;

FIG. 11 is a diagram illustrating an example functional configuration of an image processor of a modification of the first embodiment;

FIG. 12 is a diagram illustrating an example functional configuration of an image processor of a example modification of the first embodiment;

FIG. 13 is a diagram illustrating an example functional configuration of an image processor according to a second embodiment;

FIG. 14 is a schematic diagram representing a third cross-sectional image of the second embodiment;

FIG. 15 is a schematic diagram representing a first cross-sectional image of a modification of the second embodiment;

FIG. 16 is a schematic diagram representing a third cross-sectional image of a modification of the second embodiment;

FIG. 17 is a diagram illustrating an example functional configuration of an image processor according to a third embodiment;

FIG. 18 is a diagram illustrating an example functional configuration of an image processor of a modification of the third embodiment; and

FIG. 19 is a diagram illustrating an example configuration of an ultrasonic diagnostic apparatus according to another embodiment.

### DETAILED DESCRIPTION

According to an embodiment, an ultrasonic diagnostic apparatus includes a first acquirer, a first generator, a second generator, and a display controller. The first acquirer acquires a 3D ultrasonic image including a heart for one or more phases. The first generator generates a first cross-sectional image showing a cross-section of the 3D ultrasonic image which includes a first axis passing through a second portion in the 3D ultrasonic image and which includes a third portion. The second portion is a portion by which one of blood inflow into a first portion which is an atrium or a ventricle and blood outflow from the first portion is performed. The third portion is a portion by which another of the blood inflow into the first portion and the blood outflow from the first portion is performed. The second generator generates a second cross-sectional image showing a cross-section of the 3D ultrasonic image which includes a second axis passing through the third portion in the first cross-sectional image and which intersects with the first cross-sectional image. The display controller performs control of displaying the first cross-sectional image and the second cross-sectional image.

Hereinafter, various embodiments will be described in detail with reference to the appended drawings.

### First Embodiment

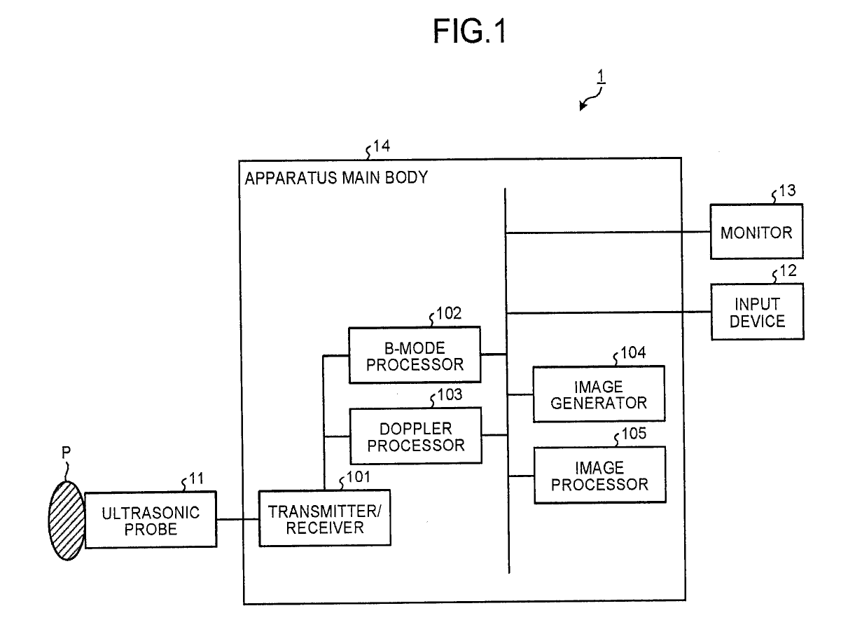

FIG. 1 is a diagram illustrating an example configuration of an ultrasonic diagnostic apparatus 1 according to a first embodiment. In the following, a case where the ultrasonic diagnostic apparatus 1 captures an image of the heart of a subject P will be described as an example. As illustrated in FIG. 1, the ultrasonic diagnostic apparatus 1 includes an ultrasonic probe 11, an input device 12, a monitor 13, and an apparatus main body 14.

The ultrasonic probe 11 includes a plurality of piezoelectric vibrators. The plurality of piezoelectric vibrators generate ultrasonic waves based on drive signals supplied from a transmitter/receiver 101 provided to the apparatus main body 14 described later, and also receive reflected waves from the subject P and convert the same into electrical signals. The ultrasonic probe 11 includes a matching layer provided to the piezoelectric vibrator, a backing member for preventing propagation of ultrasonic waves from the piezoelectric vibrator toward the back, and the like.

When an ultrasonic wave is transmitted to the subject P from the ultrasonic probe 11, the transmitted ultrasonic wave is sequentially reflected by discontinuities of acoustic impedances in the body tissues of the subject P, and reflected wave signals are received by the plurality of piezoelectric vibrators provided to the ultrasonic probe 11. The amplitude of a received reflected wave signal is dependent on the difference in the acoustic impedances at the discontinuity where the ultrasonic wave is reflected. The reflected wave signal of a case where a transmitted ultrasonic pulse is reflected by a moving blood flow, the surface of the heart wall or the like, is dependent on the velocity component of the moving object with respect to the ultrasonic wave transmission direction due to the Doppler effect, and undergoes frequency shifting.

In the first embodiment, a mechanical 4D probe as the ultrasonic probe 11 is connected to the apparatus main body 14 for the purpose of 3D scanning of the subject P, for example. The mechanical 4D probe is able to perform 3D scanning by causing the plurality of piezoelectric vibrators arranged in one line to oscillate at a predetermined angle (angle of oscillation). Moreover, as the ultrasonic probe 11 for 3D scanning, a 2D array probe including a plurality of piezoelectric vibrators arranged in a matrix may also be used.

The input device 12 is a device used by an operator (user) of the ultrasonic diagnostic apparatus 1 to input various instructions and various settings, and may be configured by a mouse, a keyboard and the like, for example. The monitor 13 is a display device for displaying various images, and may be configured by a liquid crystal panel display device, for example. The monitor 13 is capable of displaying a GUI (Graphical User Interface) for the operator of the ultrasonic diagnostic apparatus 1 to input various instructions and various settings by using the input device 12, and of displaying an ultrasonic image and the like generated by the apparatus main body 14.

The apparatus main body 14 is a device capable of generating a 3D ultrasonic image based on 3D reflected wave data received by the ultrasonic probe 11. In the following description, the 3D ultrasonic image may be referred to as “volume data”.

As illustrated in FIG. 1, the apparatus main body 14 includes a transmitter/receiver 101, a B-mode processor 102, a Doppler processor 103, an image generator 104, and an image processor 105.

In the case of performing 3D scanning of the subject P, the transmitter/receiver 101 causes a 3D ultrasonic beam to be transmitted from the ultrasonic probe 11. Then, the transmitter/receiver 101 generates 3D reflected wave data from a 3D reflected wave signal received from the ultrasonic probe 11.

The B-mode processor 102 receives the reflected wave data from the transmitter/receiver 101, and by performing logarithmic amplification, envelope detection processing or the like, generates data (B-mode data) in which the signal intensity is expressed by the brightness of luminance. The B-mode processor 102 of the first embodiment generates 3D B-mode data from 3D reflected wave data.

The Doppler processor 103 performs frequency analysis on velocity information from the reflected wave data that is received from the transmitter/receiver 101, extracts blood flow, tissue and contrast agent echo components due to the Doppler effect, and generates data (Doppler data) extracting moving object information such as average velocity, distribution, power and the like for multiple points. The Doppler processor 103 of the first embodiment generates 3D Doppler data from the 3D reflected wave data.

The image generator 104 generates a 3D ultrasonic image from the B-mode data generated by the B-mode processor 102 or the Doppler data generated by the Doppler processor 103. Specifically, the image generator 104 generates 3D B-mode image data by performing coordinate transformation on the 3D B-mode data generated by the B-mode processor 102. Further, the image generator 104 generates 3D Doppler image data by performing coordinate transformation on the 3D Doppler data generated by the Doppler processor 103. That is, the image generator 104 generates “3D B-mode image data or 3D Doppler image data” as “3D ultrasonic image (volume data)”.

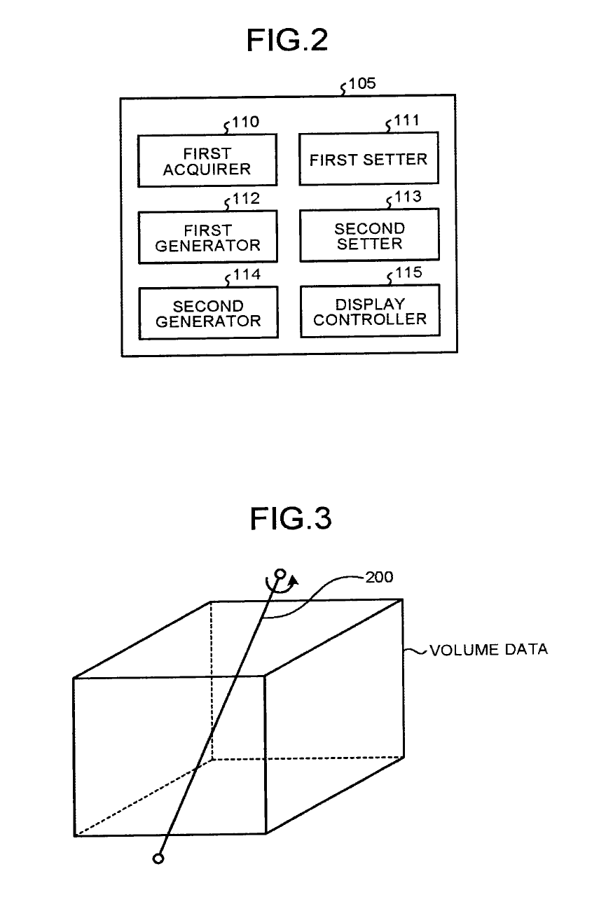

The image processor 105 performs image processing on the volume data generated by the image generator 104, and performs control of displaying an image subjected to the image processing on the monitor 13. FIG. 2 is a diagram illustrating an example functional configuration of the image processor 105 according to the first embodiment. As illustrated in FIG. 2, the image processor 105 includes a first acquirer 110, a first setter 111, a first generator 112, a second setter 113, a second generator 114, and a display controller 115.

The first acquirer 110 acquires the volume data generated by the image generator 104. In the first embodiment, a case where the volume data to be acquired by the first acquirer 110 is a still image is described as an example, but this is not restrictive. In short, the first acquirer 110 may take any mode as long as a 3D ultrasonic image including the heart at one or more phases is acquired. In the present specification, “one phase” refers to any one time point (timing) in the periodic motion of the heart. In the first embodiment, the first acquirer 110 may also acquire volume data at one phase corresponding to the end-diastole or the end-systole, for example.

The first setter 111 sets, in the volume data acquired by the first acquirer 110, a first axis passing through a second portion by which one of blood inflow into a first portion which is an atrium or the ventricle and blood outflow from the first portion is performed. In the first embodiment, description is given, as an example, of a case where the first portion is the “right ventricle” and the second portion is the “tricuspid valve (inflow portion)” for the blood to flow into the right ventricle, but this is not restrictive. Moreover, the second portion may be a tubular region, but is not limited to be the tubular region. In the first embodiment, the first setter 111 sets the first axis according to an input (operation) of a user. Details are given below.

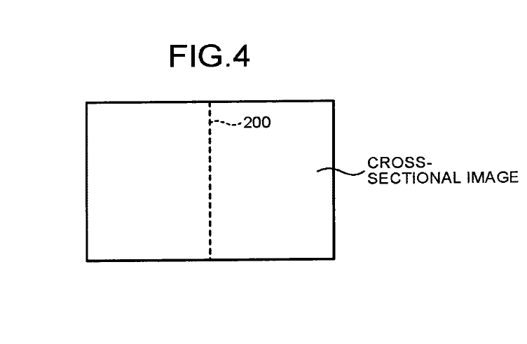

When the volume data is acquired by the first acquirer 110, a cross-sectional image that passes through an axis 200 of the volume data illustrated in FIG. 3 is displayed on the monitor 13. In this example, as illustrated in FIG. 4, the axis 200 is arranged, in the cross-sectional image displayed on the monitor 13, extending along the center portion of the cross-sectional image. In the first embodiment, a user searches for a cross-sectional image showing the tricuspid valve by switching the cross-sectional images displayed on the monitor 13 by performing an operation of changing the direction of the axis 200 or of rotating the axis 200. When the tricuspid valve is found, the user performs an operation of causing the axis 200 to pass inside the tricuspid valve (to be along the center line of the tricuspid valve), and then inputs an instruction for causing the current axis 200 to be the first axis. When this input is received, the first setter 111 sets the current axis 200 as the first axis. In this example, the first axis can be assumed to be an axis along the long axis direction of the heart.

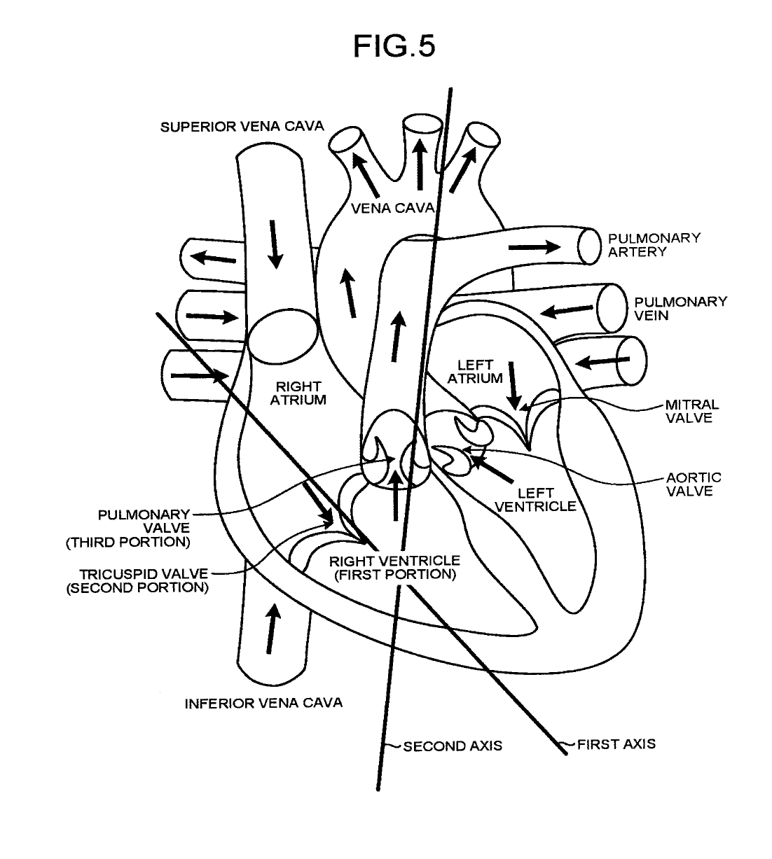

FIG. 5 is a diagram schematically illustrating the structure of the heart included in the volume data. In FIG. 5, the right ventricle, which is the first portion in the first embodiment, is located at the lowermost part of the heart, and is connected to the right atrium located at the upper right part of the heart via the tricuspid valve, which is the second portion in the first embodiment. Blood flows into the right atrium through each of the superior vena cava and the inferior vena cava. The blood which has flowed into the right atrium flows into the right ventricle through the tricuspid valve. The right ventricle is connected to the pulmonary artery via the pulmonary valve. The pulmonary valve is a valve for the blood to flow out of the right ventricle. In the first embodiment, the pulmonary valve corresponds to a third portion described later. Details will be given later. As illustrated in FIG. 5, the first axis set by the first setter 111 described above is an axis that passes through the tricuspid valve (second portion). The first axis illustrated in FIG. 5 is only an example, and is not restrictive. As illustrated in FIG. 5, a second axis described later is an axis that passes through the pulmonary valve (third portion). Specifics of the second axis will be given later. The second axis illustrated in FIG. 5 is only an example, and is not restrictive.

FIG. 2 will be described further. The first generator 112 generates a first cross-sectional image indicating a cross-section of a 3D ultrasonic image including the first axis and the third portion by which the other of the blood inflow into the first portion and the blood outflow from the first portion is performed. In the first embodiment, description is given, as an example, of a case where the third portion is the “pulmonary valve (outflow portion)” for the blood to flow out of the right ventricle, but this is not restrictive. The third portion may be a tubular region, but is not limited to be the tubular region. In the first embodiment, the first generator 112 generates the first cross-sectional image according to an input of a user. Specifics are as below.

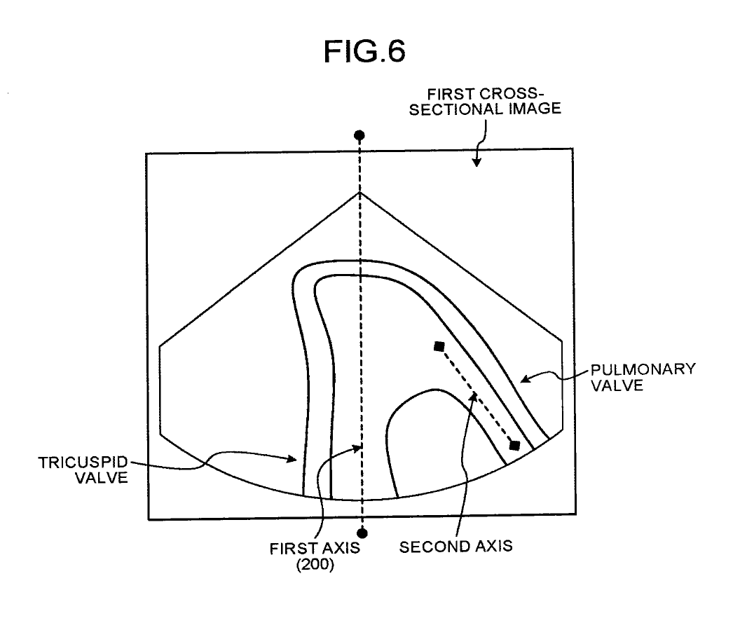

A user performs an operation of rotating the axis 200 set as the first axis by the first setter 111, and searches for a cross-sectional image showing the pulmonary valve by switching the cross-sectional images displayed on the monitor 13. Then, when a cross-sectional image showing the pulmonary valve is found, an instruction for causing the current cross-sectional image to be the first cross-sectional image is input. The first generator 112 which has received this input generates (sets) the current cross-sectional image as the first cross-sectional image. The first cross-sectional image represents a cross-section of a part of the heart illustrated in FIG. 5. FIG. 6 is a diagram illustrating an example of the first cross-sectional image of the first embodiment. The first cross-sectional image of the first embodiment is a cross-section of volume data including the first axis passing through the tricuspid valve (second portion) and the pulmonary valve.

FIG. 2 will be described further. The second setter 113 sets the second axis that passes through the third portion in the first cross-sectional image. In the first embodiment, the second setter 113 sets the second axis according to an input of a user. More specifically, a user performs an operation of setting an axis along the center line of the pulmonary valve shown in the first cross-sectional image as the second axis. Then, the second setter 113 sets the second axis according to this operation by the user.

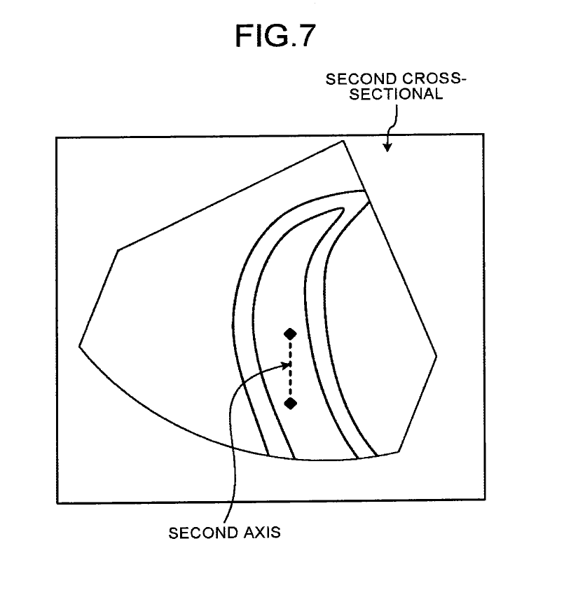

The second generator 114 generates a second cross-sectional image showing a cross-section (cross-section of volume data) which includes the second axis and which crosses the first cross-sectional image. For example, the second generator 114 may generate, as the second cross-sectional image, a cross-section of the volume data which includes the second axis set by the second setter 113 and which is orthogonal to the first cross-sectional image generated by the first generator 112. The second cross-sectional image represents a cross-section of a part of the heart illustrated in FIG. 5. FIG. 7 is a diagram illustrating an example of the second cross-section of the first embodiment. The second cross-sectional image of the first embodiment is a cross-section of volume data which includes the second axis passing through the pulmonary valve (third portion) in the first cross-sectional image illustrated in FIG. 6, and which is orthogonal to the first cross-sectional image illustrated in FIG. 6.

FIG. 2 will be described further. The display controller 115 performs control of displaying the first cross-sectional image and the second cross-sectional image. More specifically, the display controller 115 performs control of displaying, on the monitor 13 (an example of a display for displaying an image), the first cross-sectional image generated by the first generator 112 and the second cross-sectional image generated by the second generator 114. For example, as illustrated in FIGS. 6 and 7, the display controller 115 may also perform control of displaying, on each of the first cross-sectional image and the second cross-sectional image, information indicating the first axis and information indicating the second axis. However, this is not restrictive, and the display controller 115 may perform control of displaying the first cross-sectional image and the second cross-sectional image without displaying the information indicating the first axis and the information indicating the second axis.

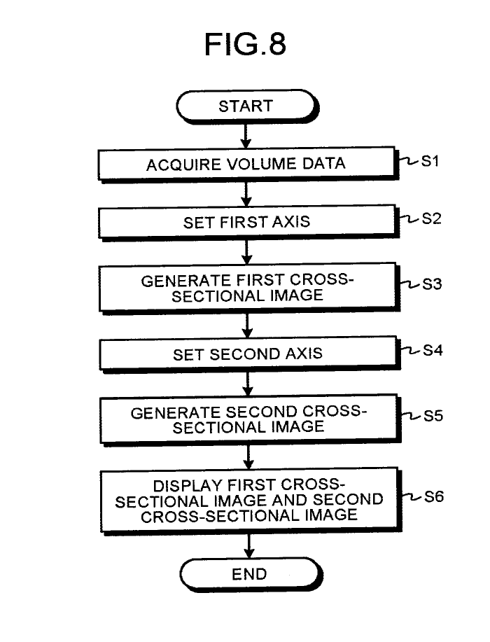

FIG. 8 is a flowchart illustrating example processing performed by the image processor 105 of the first embodiment. As illustrated in FIG. 8, the first acquirer 110 acquires volume data (Step S1). The first setter 111 sets a first axis (Step S2). The first generator 112 generates a first cross-sectional image (Step S3). The second setter 113 sets a second axis (Step S4). The second generator 114 generates a second cross-sectional image (Step S5). Then, the display controller 115 displays the first cross-sectional image and the second cross-sectional image (Step S6). Specifics on the steps are as described above.

In the case of capturing an image of the subject P by setting the position of the ultrasonic probe 11 according to the axis that passes through the right ventricle and the tricuspid valve (inflow portion), the pulmonary valve (outflow portion) of the right ventricle is drawn unclearly due to the restriction of an acoustic window (an intercostal region, not overlapping the lungs, where an ultrasonic wave may pass), and there is an apparent problem that it is difficult to visually check the myocardial boundary using only the short-axis cross-section, as with the conventional technique. As exemplified by an apical four-chamber view, to cover all of the left ventricle and the right ventricle, an acoustic window by an apical approach is used. As in an apical two-chamber view or an apical long-axis view which is further obtained by this approach, the left ventricle may be drawn as a 2D tomographic image. However, with the right ventricle, the inflow side and the outflow side cannot be drawn at the same time as a 2D tomographic image. Thus, to obtain a cross-section of the right ventricle in the manner of the first cross-sectional image, volume data has to be collected and reconstructed. At this time, since the aorta on the left side is located on the inner body side than the pulmonary artery (blood through which an ultrasonic wave easily passes) on the right side, and the pulmonary artery is present between the aorta and the superior mediastinum, which is a bone, and the lungs near the pulmonary valve, it is not easily affected by the side lobes thereof. On the other hand, the pulmonary artery, which is close to the body surface side, is close to the bones and the lungs, and thus, in the arrangement of the first cross-sectional image, it is easily affected by the side lobe in the azimuth direction, and the image quality is reduced. Accordingly, it is often difficult to set the second axis using the first cross-sectional image.

Accordingly, in the first embodiment, the first cross-sectional image showing the cross-section of volume data which passes through the first axis along the center line of the tricuspid valve and which includes the pulmonary valve, and the second cross-sectional image which passes through the second axis along the center line of the pulmonary valve shown in the first cross-sectional image and which crosses the first cross-sectional image are generated and displayed on the monitor 13. With the second cross-sectional image, by selecting an arrangement, with respect to the outflow portion, according to which the cardiac muscle tissue on the anterior wall side of the left ventricle or the blood in the left chamber of the heart is present between the outflow portion and the lungs or according to which the outflow portion is located between the bones and the lungs and is not in direct contact with these, the influence of the side lobes of the lungs and bones are relatively reduced, and the image quality is improved. By reconstructing and drawing a cross-section with highly visible arrangement as described above by using the volume data, the visibility of the pulmonary valve which was conventionally difficult to see due to the restriction of the acoustic window may be increased, and the user is enabled to set the myocardial boundary with ease and high accuracy. With the setting of the myocardial boundary facilitated, the time required for the analysis and the diagnosis may be reduced. Further, with the increase in the accuracy of setting of the myocardial boundary, the accuracy of the analysis and the diagnosis is also increased. Accordingly, with the first embodiment, both reduction in the time required for the analysis and diagnosis and increase in the accuracy of the analysis and the diagnosis may be achieved.

Hardware Configuration and Program The hardware configuration of the apparatus main body 14 in which the image processor 105 described above is mounted uses the hardware configuration of a computer device including a CPU (Central Processing Unit), ROM, RAM, a communication I/F device and the like. The function of each unit (transmitter/receiver 101, B-mode processor 102, Doppler processor 103, image generator 104, image processor 105 (first acquirer 110, first setter 111, first generator 112, second setter 113, second generator 114, display controller 115)) of the apparatus main body 14 described above is implemented by the CPU loading a program stored in the ROM into the RAM. Furthermore, it is also possible to implement at least a part of the functions of the units of the apparatus main body 14 described above by a dedicated hardware circuit (for example, a semiconductor integrated circuit or the like).

In the first embodiment, the apparatus main body 14 installed with the function of the image processor 105 described above is assumed to correspond to an “image processing apparatus” in the claims.

The programs to be executed by the CPU (computer) described above may be stored in an external device connected to a network such as the Internet, and may be provided by being downloaded via the network. Furthermore, the programs to be executed by the CPU described above may be provided or distributed via the network such as the Internet. Moreover, the programs to be executed by the CPU described above may be provided being embedded in advance in a non-volatile recording medium such as the ROM.

### First Modification of First Embodiment

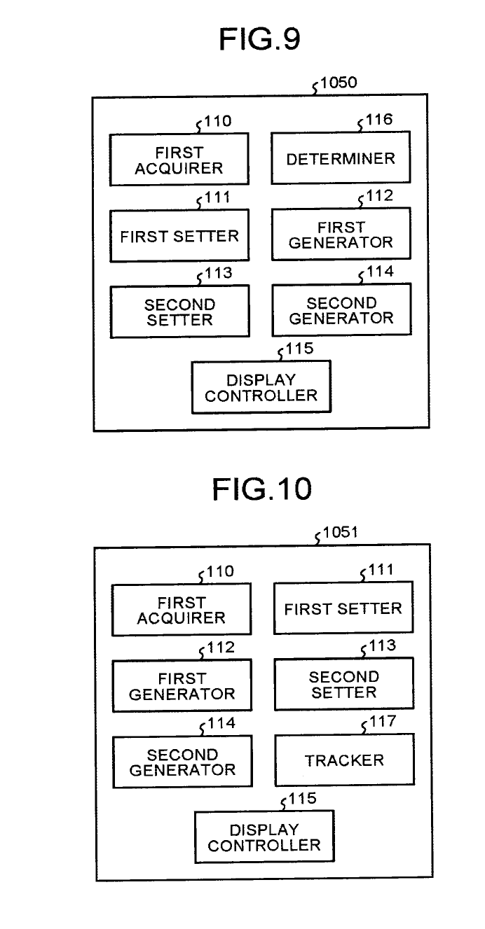

For example, the first axis, the first cross-sectional image and the second axis may be automatically set by pattern recognition. FIG. 9 is a diagram illustrating an example functional configuration of an image processor 1050 of a first modification. As illustrated in FIG. 9, the image processor 1050 further includes a determiner 116. The determiner 116 compares each piece of candidate data with dictionary data. The each piece of candidate data indicates a combination of a candidate for the first axis, a candidate for the first cross-sectional image, and a candidate for the second axis of the volume data acquired by the first acquirer 110. The dictionary date indicates the volume data in which a positional relationship of the first axis, the first cross-sectional image, and the second axis is determined in advance. The determiner 116 then determines a maximum combination indicating a combination of the candidates with a highest degree of coincidence with the dictionary data. The comparison is performed after performing position alignment.

The first setter 111 arbitrarily sets a plurality of candidates for the first axis for the volume data acquired by the first acquirer 110. Then, the first setter 111 sets the candidate, for the first axis, included in the maximum combination determined by the determiner 116 as the first axis.

The first generator 112 arbitrarily sets a plurality of candidates for the first cross-sectional image for the volume data acquired by the first acquirer 110. Then, the first generator 112 sets the candidate, for the first cross-sectional image, included in the maximum combination determined by the determiner 116 as the first cross-sectional image.

Moreover, the second setter 113 arbitrarily sets a plurality of candidates for the second axis for the volume data acquired by the first acquirer 110. Then, the second setter 113 sets the candidate, for the second axis, included in the maximum combination determined by the determiner 116 as the second axis.

### Second Modification of First Embodiment

For example, the first acquirer 110 may acquire volume data at two or more phases. In a second modification, after the first axis and the second axis are set for the volume data at a predetermined phase, the positions of the first axis and the second axis at a phase (one or more phases) different from the one predetermined phase are tracked by using a known 3D tracking technique using a volume data group along a time series, and the first cross-sectional image and the second cross-sectional image at the phase different from the predetermined phase are generated using the tracking result. Then, a plurality of first cross-sectional images and second cross-sectional images corresponding one-to-one with a plurality of phases are successively displayed (the first cross-sectional images and the second cross-sectional images are reproduced as a video). Specifics are as below.

FIG. 10 is a diagram illustrating an example functional configuration of an image processor 1051 of the second modification. As illustrated in FIG. 10, the image processor 1051 further includes a tracker 117. By taking the first axis and the second axis set for the volume data at a predetermined phase as initial positions, the tracker 117 estimates the positions of the first axis and the second axis in the volume data at a phase different from the predetermined phase by tracking the initial positions based on motion information at the phase different from the predetermined phase.

For example, in the case where a one-heartbeat section from the first end-diastole to the next end-diastole is set as a tracking target section, a phase corresponding to the first end-diastole may be taken as the predetermined phase described above. In this case, the tracker 117 may estimate the positions of the first axis and the second axis in the volume data at each of a plurality of phases (remaining phases) in the one-heartbeat section other than the phase corresponding to the first end-diastole. Alternatively, for example, in the case where a section from the first end-systole to the next end-systole is set as the tracking target section, a phase corresponding to the first end-systole may be taken as the predetermined phase described above. Still alternatively, a plurality of heartbeat sections may be set as the tracking target section, for example.

For example, the tracker 117 may estimate the positions of the first axis and the second axis in volume data at a phase that is temporally adjacent to a predetermined phase by estimating the motion information between volume data at the predetermined phase and volume data at the phase (an example of the phase that is different from the predetermined phase) that is temporally adjacent to the predetermined phase, and moving the first axis and the second axis that are set for the volume data at the predetermined phase based on the estimated motion information. As the estimation method for the motion information, various known techniques such as local pattern matching processing, an optical flow method and the like may be used.

The first generator 112 generates the first cross-sectional image at a phase that is different from the predetermined phase based on the position of the first axis tracked by the tracker 117 (the first cross-sectional image is generated for each of the one or more phases). The second generator 114 generates the second first cross-sectional image at a phase that is different from the predetermined phase based on the position of the second axis tracked by the tracker 117 (the second cross-sectional image is generated for each of the one or more phases).

Then, the display controller 115 performs control of successively displaying a plurality of first cross-sectional images and second cross-sectional images corresponding one-to-one with a plurality of phases.

For example, the display controller 115 may perform control of successively displaying a plurality of first cross-sectional images and second cross-sectional images corresponding one-to-one with all the phases included in a tracking target section. Alternatively, for example, the display controller 115 may alternately display the first cross-sectional image and the second cross-sectional image corresponding to a phase at the end-diastole in a heartbeat section and the first cross-sectional image and the second cross-sectional image corresponding to a phase at the end-systole in the heartbeat section. Moreover, for example, in the case where a plurality of heartbeat sections are set as the tracking target section, the display controller 115 may perform control of successively displaying the first cross-sectional image and the second cross-sectional image corresponding to a phase at the end-diastole in the first heartbeat section and the first cross-sectional image and the second cross-sectional image corresponding to a phase at the end-systole in the first heartbeat section, and then successively displaying the first cross-sectional image and the second cross-sectional image corresponding to a phase at the end-diastole in the second heartbeat section immediately following the first heartbeat section and the first cross-sectional image and the second cross-sectional image corresponding to a phase at the end-systole in the second heartbeat section.

Furthermore, for example, the display controller 115 may perform control of alternately displaying the first cross-sectional image and the second cross-sectional image corresponding to a phase (in the following description, sometimes referred to as “target phase”) in a tracking target section and the first cross-sectional image and the second cross-sectional image corresponding to a phase preceding or following the target phase.

### Third Modification of First Embodiment

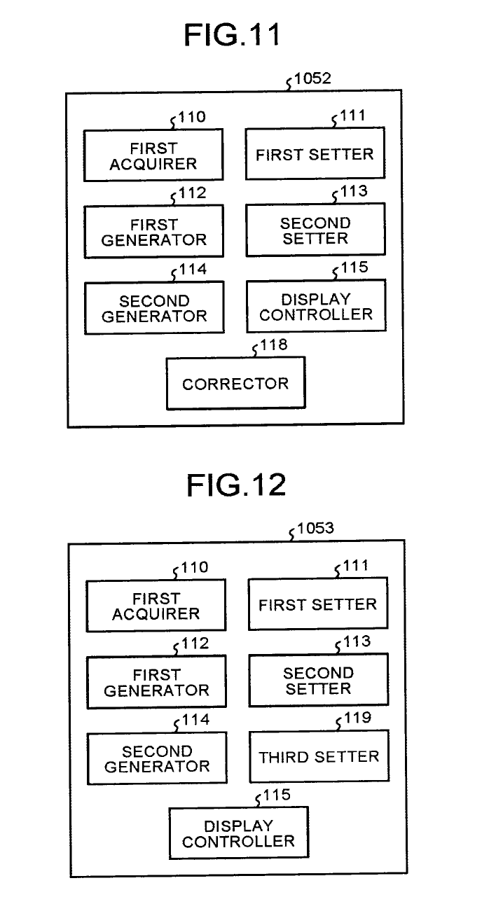

A function may also be included for correcting the position of the first axis or the second axis according to an input of a user, and changing the first cross-sectional image or the second cross-sectional image according to the correction. FIG. 11 is a diagram illustrating an example functional configuration of an image processor 1052 of a third modification. As illustrated in FIG. 11, the image processor 1052 further includes a corrector 118. The corrector 118 corrects the position of the first axis or the second axis according to an input of a user, and changes the first cross-sectional image or the second cross-sectional image according to the correction.

In the case where a user performs an operation on the first axis, as illustrated in FIG. 6, for example, which is displayed in the first cross-sectional image, to change the direction of the first axis, the corrector 118 corrects the position of the first axis according to the operation, and changes the first cross-sectional image according to the correction. In a similar manner, in the case where the user performs an operation on the second axis, as illustrated in FIG. 6, for example, which is displayed in the first cross-sectional image, to change the direction of the second axis, the corrector 118 corrects the position of the second axis according to the operation, and changes the second cross-sectional image according to the correction.

### Fourth Modification of First Embodiment

For example, a function may also be included for setting the diameter of the third portion (in this example, the pulmonary valve), which is a tubular portion, by using a second cross-sectional image. Description is given here of case where the third portion is a tubular region as an example, but the third portion is not limited to be a tubular region. FIG. 12 is a diagram illustrating an example functional configuration of an image processor 1053 of a fourth modification. As illustrated in FIG. 12, the image processor 1053 further includes a third setter 119. The third setter 119 sets the diameter of the pulmonary valve by using a second cross-sectional image generated by the second generator 114. Then, for example, the display controller 115 may display information indicating the diameter of the pulmonary valve set by the third setter 119 in the first cross-sectional image or the second cross-sectional image.

### Second Embodiment

Next, a second embodiment will be described. In the second embodiment, a function of generating a third cross-sectional image which is a cross-section in the short-axis direction of the heart included in volume data and which includes a first portion (in this example, the right ventricle) is further included. Specifics will be given below. Parts common with the first embodiment described above will be omitted from the description as appropriate.

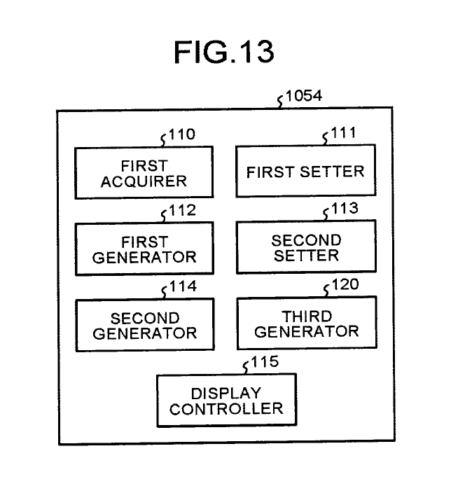

FIG. 13 is a diagram illustrating an example functional configuration of an image processor 1053 according to the second embodiment. As illustrated in FIG. 13, the image processor 1053 further includes a third generator 120. The third generator 120 generates a third cross-sectional image which is a cross-section in the short-axis direction of the heart included in volume data and which includes the right ventricle. For example, the third generator 120 may generate (set), as the third cross-sectional image, a cross-section which is orthogonal to a first axis which is an axis in the long-axis direction of the heart and which is of volume data including the right ventricle, or may generate (set), as the third cross-sectional image, a cross-section which is orthogonal to an axis connecting an apex portion in the volume data recognized by pattern matching or the like and the tricuspid valve (an axis in the long-axis direction of the heart) and which is of volume data including the right ventricle.

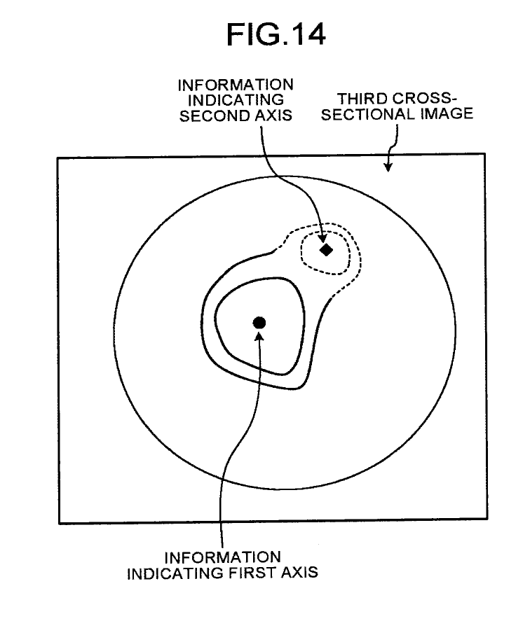

Then, as illustrated in FIG. 14, the display controller 115 performs control of displaying, in the third cross-sectional image, information indicating a first axis (in the example in FIG. 14, a mark representing the first axis) and information indicating a second axis (in the example in FIG. 14, a mark representing the second axis). As described above, in the second embodiment, with information indicating the first axis and information indicating the second axis being displayed in the third cross-sectional image which is a cross-section in the short-axis direction, in addition to the first cross-sectional image and the second cross-sectional image, a user is enabled to check the myocardial boundary with ease and high accuracy.

### Modification of Second Embodiment

For example, the display controller 115 may also perform control of displaying, according to an input of a user, boundary information indicating the boundary of the first portion (in this example, the right ventricle) in the first cross-sectional image or the second cross-sectional image, and information indicating the position corresponding to the boundary information in the third cross-sectional image.

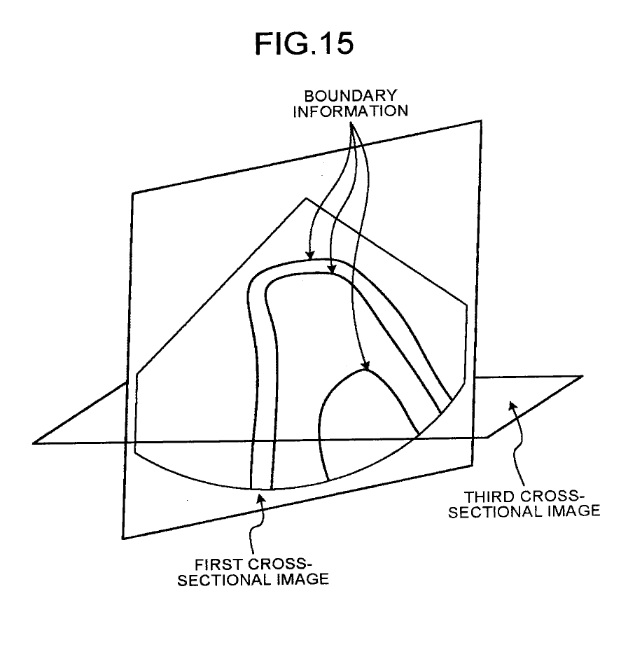

In this example, the boundary information is information indicating the myocardial boundary of the right ventricle, and for example, the display controller 115 may generate the boundary information indicating the myocardial boundary by connecting a dot sequence input by the user in the first cross-sectional image, and may superimpose and display the generated boundary information on the first cross-sectional image, as illustrated in FIG. 15. As the generation method of boundary information, various known techniques may be used, and for example, the boundary information indicating the myocardial boundary may be generated based on a curve input by a user using a pen-type input device.

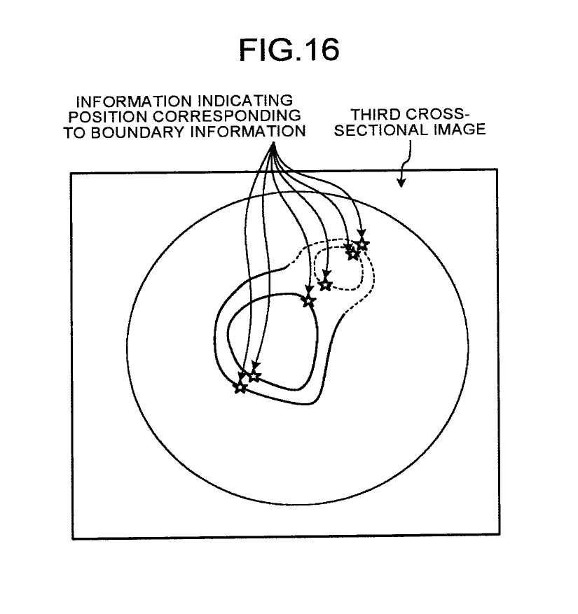

Then, as illustrated in FIG. 16, the display controller 115 performs control of superimposing and displaying, in the third cross-sectional image, information indicating the position corresponding to the boundary information.

### Third Embodiment

Next, a third embodiment will be described. In the third embodiment, a function of acquiring 3D-shape information indicating the 3D shape of a first portion (in this example, the right ventricle) is further included, and the display controller 115 performs control of displaying the 3D-shape information in each of the first cross-sectional image and the second cross-sectional image. Specifics will be given below. Parts common with the first embodiment described above will be omitted from the description as appropriate.

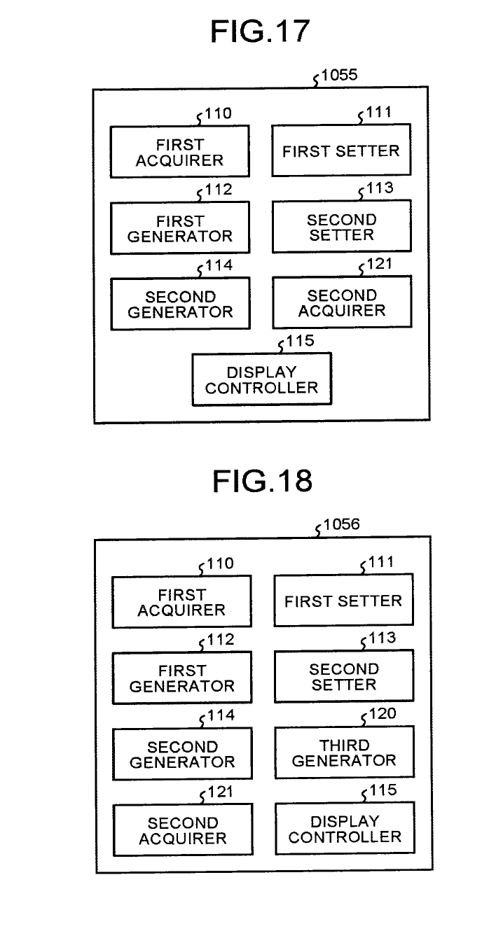

FIG. 17 is a diagram illustrating an example functional configuration of an image processor 1055 according to the third embodiment. As illustrated in FIG. 17, the image processor 1055 further includes a second acquirer 121. In this example, the second acquirer 121 acquires (reads) 3D-shape information indicating the myocardial boundary of the right ventricle from an external device not illustrated (for example, a server or a memory). This 3D-shape information may express the myocardial boundary of the right ventricle as a dot sequence, or it may be 3D label data.

Then, the display controller 115 performs control of superimposing and displaying the 3D-shape information acquired by the second acquirer 121 in each of the first cross-sectional image and the second cross-sectional image. In this example, the display controller 115 may also perform control of displaying, in each of the first cross-sectional image and the second cross-sectional image, information (for example, a mark) indicating the position that intersects the myocardial boundary of the right ventricle indicated by the 3D-shape information acquired by the second acquirer 121. As described above, according to the third embodiment, a user may check the myocardial boundary of the right ventricle in a cross-sectional image (first cross-sectional image, second cross-sectional image) with increased visibility of the pulmonary valve (the outflow portion of the right ventricle).

### Modification of Third Embodiment

For example, a function may further be included for generating a third cross-sectional image which is a cross-section in the short-axis direction of the heart included in volume data and which includes a first portion (in this example, the right ventricle), and the display controller 115 may display the 3D-shape information in the third cross-sectional image. FIG. 18 is a diagram illustrating an example functional configuration of an image processor 1056 of the modification. As illustrated in FIG. 18, the image processor 1056 further includes the third generator 120 described above. In the modification, the display controller 115 performs control of displaying the 3D-shape information acquired by the second acquirer 121 in the third cross-sectional image.

### Another Embodiment

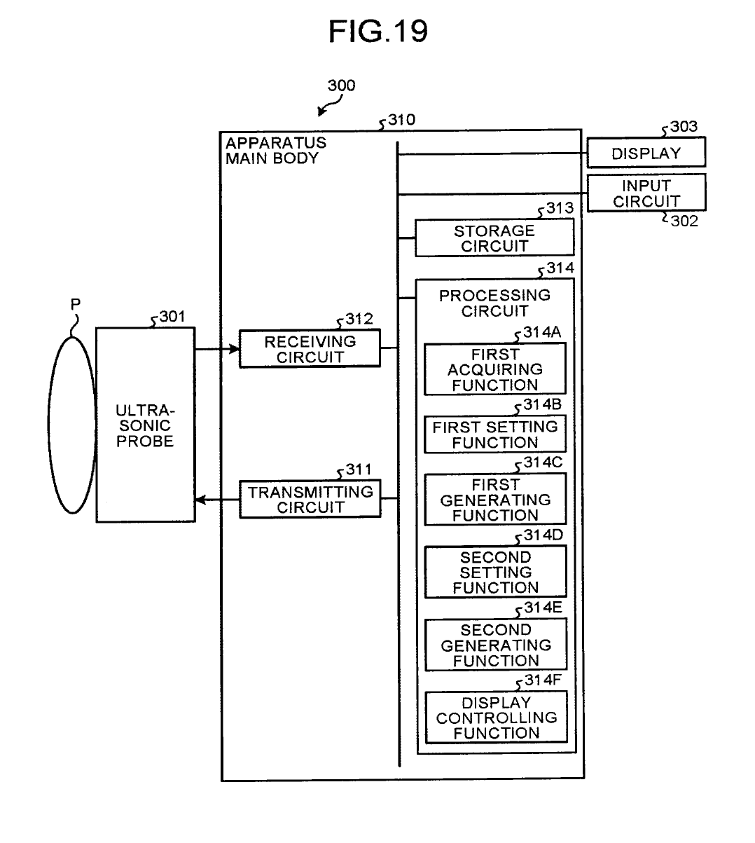

For example, the ultrasonic diagnostic apparatus 1 illustrated in FIG. 1 may be configured as illustrated in FIG. 19. FIG. 19 is a block diagram illustrating an example configuration of an ultrasonic diagnostic apparatus 300 according to another embodiment. As illustrated in FIG. 19, the ultrasonic diagnostic apparatus 300 includes an ultrasonic probe 301, an input circuit 302, a display 303, and an apparatus main body 310. The ultrasonic probe 301, the input circuit 302, and the display 303 correspond to the ultrasonic probe 11, the input device 12, and the monitor 13 illustrated in FIG. 1, respectively.

The apparatus main body 310 includes a transmitting circuit 311, a receiving circuit 312, a storage circuit 313, and a processing circuit 314. The transmitting circuit 311 and the receiving circuit 312 correspond to the transmitter/receiver 101 illustrated in FIG. 1. The storage circuit 313 stores therein a variety of information such as a program executed by the processing circuit 314.

The processing circuit 314 corresponds to the B-mode processor 102, the Doppler processor 103, the image generator 104, and the image processor 105 illustrated in FIG. 1. That is, the processing circuit 314 performs processing performed by the B-mode processor 102, the Doppler processor 103, the image generator 104, and the image processor 105. The processing circuit 314 is an example processing circuit in the accompanying claims.

The processing circuit 314 performs a first acquiring function 314A, a first setting function 314B, a first generating function 314C, a second setting function 314D, a second generating function 314E, and a display controlling function 314F. The first acquiring function 314A is a function implemented by the first acquirer 110 illustrated in FIG. 2. The first setting function 314B is a function implemented by the first setter 111 illustrated in FIG. 2. The first generating function 314C is a function implemented by the first generator 112 illustrated in FIG. 2. The second setting function 314D is a function implemented by the second setter 113 illustrated in FIG. 2. The second generating function 314E is a function implemented by the second generator 114 illustrated in FIG. 2. The display controlling function 314F is a function implemented by the display controller 115 illustrated in FIG. 2.

For example, each of the respective processing functions performed by the first acquiring function 314A, the first setting function 314B, the first generating function 314C, the second setting function 314D, the second generating function 314E, and the display controlling function 314F, which are components of the processing circuit 314 illustrated in FIG. 19, is stored in the storage circuit 313 in a form of a computer-executable program. The processing circuit 314 is a processor that reads and executes programs from the storage circuit 313 so as to implement the respective functions corresponding to the programs. In other words, the processing circuit 314 with the programs being read has the functions illustrated in the processing circuit 314 in FIG. 19. That is, the processing circuit 314 reads and executes the program corresponding to the first acquiring function 314A from the storage circuit 313 so as to perform the same function as the first acquirer 110. The processing circuit 314 reads and executes the program corresponding to the first setting function 314B from the storage circuit 313 so as to perform the same function as the first setter 111. The processing circuit 314 reads and executes the program corresponding to the first generating function 314C from the storage circuit 313 so as to perform the same function as the first generator 112. The processing circuit 314 reads and executes the program corresponding to the second setting function 314D from the storage circuit 313 so as to perform the same function as the second setter 113. The processing circuit 314 reads and executes the program corresponding to the second generating function 314E from the storage circuit 313 so as to perform the same function as the second generator 114. The processing circuit 314 reads and executes the program corresponding to the display controlling function 314F from the storage circuit 313 so as to perform the same function as the display controller 115.

For example, Step S1 illustrated in FIG. 8 is a step implemented by causing the processing circuit 314 to read and execute the program corresponding to the first acquiring function 314A from the storage circuit 313. Step S2 illustrated in FIG. 8 is a step implemented by causing the processing circuit 314 to read and execute the program corresponding to the first setting function 314B from the storage circuit 313. Step S3 illustrated in FIG. 8 is a step implemented by causing the processing circuit 314 to read and execute the program corresponding to the first generating function 314C from the storage circuit 313. Step S4 illustrated in FIG. 8 is a step implemented by causing the processing circuit 314 to read and execute the program corresponding to the second setting function 314D from the storage circuit 313. Step S5 illustrated in FIG. 8 is a step implemented by causing the processing circuit 314 to read and execute the program corresponding to the second generating function 314E from the storage circuit 313. Step S6 illustrated in FIG. 8 is a step implemented by causing the processing circuit 314 to read and execute the program corresponding to the display controlling function 314F from the storage circuit 313.

In FIG. 19, the description has been given of a case where the single processing circuit 314 implements each of the respective processing functions performed by the first acquiring function 314A, the first setting function 314B, the first generating function 314C, the second setting function 314D, the second generating function 314E, and the display controlling function 314F. A plurality of separate processors may, however, be combined to form a processing circuit and the processors may execute programs so as to implement functions.

The term “processor” used in the above description, for example, refers to a central preprocess unit (CPU), a graphics processing unit (GPU), or a circuit such as an application specific integrated circuit (ASIC), a programmable logic device (for example, a simple programmable logic device (SPLD)), a complex programmable logic device (CPLD), and a field programmable gate array (FPGA)). The processor reads and executes a program stored in a storage circuit so as to implement a function. The program may be built directly in a circuit of the processor instead of being stored in a storage circuit. In this case, the processor reads and executes the program built in the circuit so as to implement a function. A configuration of the processors in the present embodiment is not limited to a case in which each of the processors is configured as a single circuit. A plurality of separate circuits may be combined into one processor that implements the respective functions. Furthermore, the components in FIG. 19 may be integrated into one processor that implements the respective functions.

The circuits exemplified in FIG. 19 may he configured in a distributed or integrated manner as appropriate. For example, the processing circuit 314 may be configured to be distributed over a circuit having a function of the B-mode processor 102, a circuit having a function of the Doppler processor 103, and a circuit having functions of the an image generator 104 and the image processor 105.

The embodiments and the modifications described above may be arbitrarily combined.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
processing circuitry configured to serve as
a first acquirer to acquire a 3D ultrasonic image including a heart for one or more phases;
a first generator to generate a first cross-sectional image showing a cross-section of the 3D ultrasonic image which includes a first axis passing through a second portion in the 3D ultrasonic image and which includes a third portion, the second portion being a portion by which one of blood inflow into a first portion which is an atrium or a ventricle and blood outflow from the first portion is performed, and the third portion being a portion by which another of the blood inflow into the first portion and the blood outflow from the first portion is performed;
a second generator to generate a second cross-sectional image showing a cross-section of the 3D ultrasonic image which includes a second axis passing through the third portion in the first cross-sectional image, and which intersects with the first cross-sectional image; and
a display controller to perform control of displaying the first cross-sectional image and the second cross-sectional image.

2. The apparatus according to claim 1, wherein
the processing circuitry is further configured to serve as
a first setter to set the first axis; and
a second setter to set the second axis.

3. The apparatus according to claim 2, wherein
the first setter sets the first axis according to an input of a user, and
the second setter sets the second axis according to the input of the user.

4. The apparatus according to claim 2, wherein
the processing circuitry is further configured to serve as a determiner to
compare each piece of candidate data with dictionary data, the each piece of candidate data indicating a combination of a candidate for the first axis, a candidate for the first cross-sectional image, and a candidate for the second axis of the 3D ultrasonic image acquired by the first acquirer, and the dictionary date indicating the 3D ultrasonic image in which a positional relationship of the first axis, the first cross-sectional image, and the second axis is determined in advance, and
determine a maximum combination indicating a combination of the candidates with a highest degree of coincidence with the dictionary data,
the first setter sets the candidate for the first axis included in the maximum combination as the first axis,
the first generator sets the candidate for the first cross-sectional image included in the maximum combination as the first cross-sectional image, and
the second setter sets the candidate for the second axis included in the maximum combination as the second axis.

5. The apparatus according to claim 4, wherein
the first setter sets a plurality of candidates for the first axis for the 3D ultrasonic image acquired by the first acquirer,
the first generator sets a plurality of candidates for the first cross-sectional image for the 3D ultrasonic image acquired by the first acquirer, and
the second setter sets a plurality of candidates for the second axis for the 3D ultrasonic image acquired by the first acquirer.

6. The apparatus according to claim 1, wherein the display controller performs control of displaying, in each of the first cross-sectional image and the second cross-sectional image, information indicating the first axis or information indicating the second axis.

7. The apparatus according to claim 1, wherein
the processing circuitry is further configured to serve as a corrector to
correct, according to an input of a user, a position of the first axis or the second axis, and
change the first cross-sectional image or the second cross-sectional image according to the correction.

8. The apparatus according to claim 1, wherein
the processing circuitry is further configured to serve as a third generator to generate a third cross-sectional image which is a cross-section in a short-axis direction of the heart included in the 3D ultrasonic image and which includes the first portion, and
the display controller performs control of displaying, in the third cross-sectional image, information indicating the first axis and information indicating the second axis.

9. The apparatus according to claim 8, wherein the display controller performs control of
displaying, in the first cross-sectional image or the second cross-sectional image, according to an input of a user, boundary information indicating a boundary of the first portion, and
displaying, in the third cross-sectional image, information indicating a position corresponding to the boundary information.

10. The apparatus according claim 1, wherein
the processing circuitry is further configured to serve as a second acquirer to acquire 3D-shape information indicating 3D shape of the first portion, and
the display controller performs control of displaying the 3D-shape information in each of the first cross-sectional image and the second cross-sectional image.

11. The apparatus according to claim 10, wherein
the processing circuitry is further configured to serve as a third generator to generate a third cross-sectional image which is a cross-section in a short-axis direction of the heart included in the 3D ultrasonic image and which includes the first portion, and
the display controller performs control of displaying the 3D-shape information in the third cross-sectional image.

12. The apparatus according to claim 1, wherein
the first portion is a right ventricle,
the second portion is a tricuspid valve, and
the third portion is a pulmonary valve.

13. The apparatus according to claim 1, wherein the first acquirer acquires 3D ultrasonic images at two or more phases.

14. The apparatus according to claim 13, wherein
the processing circuitry is further configured to serve as a tracker to take the first axis and the second axis set for the 3D ultrasonic image at a predetermined phase as initial positions thereof and track the initial positions based on motion information at a phase different from the predetermined phase, so as estimate positions of the first axis and the second axis at the phase different from the predetermined phase,
the first generator generates the first cross-sectional image at the phase different from the predetermined phase based on the position of the first axis tracked by the tracker,
the second generator generates the second cross-sectional image at the phase different from the predetermined phase based on the position of the second axis tracked by the tracker, and
the display controller performs control of successively displaying a plurality of first cross-sectional images and second cross-sectional images corresponding one-to-one with a plurality of phases.

15. The apparatus according to claim 1, wherein
the processing circuitry is further configured to serve as a third setter to set a diameter of the third portion which is a tubular portion by using the second cross-sectional image, and
the display controller performs control of displaying, in the first cross-sectional image or the second cross-sectional image, information indicating the diameter of the third portion.

16. An image processing apparatus comprising:
processing circuitry configured to serve as
a first acquirer to acquire a 3D ultrasonic image including a heart for one or more phases;
a first generator to generate a first cross-sectional image showing a cross-section of the 3D ultrasonic image which includes a first axis passing through a second portion in the 3D ultrasonic image and which includes a third portion, the second portion being a portion by which one of blood inflow into a first portion which is an atrium or a ventricle and blood outflow from the first portion is performed, and the third portion being a portion by which another of the blood inflow into the first portion and the blood outflow from the first portion is performed;
a second generator to generate a second cross-sectional image showing a cross-section of the 3D ultrasonic image which includes a second axis passing through the third portion in the first cross-sectional image, and which intersects with the first cross-sectional image; and
a display controller configured to perform control of displaying the first cross-sectional image and the second cross-sectional image.

17. An image processing method comprising:
acquiring a 3D ultrasonic image including a heart for one or more phases;
generating a first cross-sectional image showing a cross-section of the 3D ultrasonic image which includes a first axis passing through a second portion in the 3D ultrasonic image and which includes a third portion, the second portion being a portion by which one of blood inflow into a first portion which is an atrium or a ventricle and blood outflow from the first portion is performed, and the third portion being a portion by which another of the blood inflow into the first portion and the blood outflow from the first portion is performed;
generating a second cross-sectional image showing a cross-section of the 3D ultrasonic image which includes a second axis passing through the third portion in the first cross-sectional image, and which intersects with the first cross-sectional image; and
performing control of displaying the first cross-sectional image and the second cross-sectional image.
